(19) United States
(12) Patent Application Publication
(10) Pub. No.: US 2015/0250934 A1
(43) Pub. Date: Sep. 10, 2015

(54) Subject-Specific Artificial Organs and Methods for Making the Same

(71) Applicants: James K. Min, New York, NY (US); Guanglei Xiong, Irvington, NY (US)

(72) Inventors: James K. Min, New York, NY (US); Guanglei Xiong, Irvington, NY (US)

(21) Appl. No.: 14/642,625

(22) Filed: Mar. 9, 2015

Related U.S. Application Data
(60) Provisional application No. 61/949,826, filed on Mar. 7, 2014.

(51) Int. Cl. A61M 1/10; B29C 67/00; G05B 15/02; A61F 2/24

(52) U.S. Cl. CPC A61M 1/1053; A61F 2/24; A61M 1/106; B29C 67/0088; G05B 15/02; A61M 2205/10; A61M 2207/00; A61M 2207/10; B33Y 50/02

(57) ABSTRACT

An artificial heart includes an anatomically correct or patient-specific model of a natural heart, in which the model is substantially composed of an elastomer, and includes an actuation element, in which the actuation element is configured to, during operation of the artificial heart, cause the artificial heart to contract and relax.

### CROSS-REFERENCE TO RELATED APPLICATIONS

This application claims priority to Provisional Application No. 61/949,826, filed on Mar. 7, 2014, the contents of which are hereby incorporated by reference in their entirety.

### TECHNICAL FIELD

The present disclosure relates to artificial hearts and methods of making the same.

### BACKGROUND

An artificial heart is intended to replicate the function of a natural heart so that it may replace a patient's diseased or damaged heart, or be utilized to enhance the diagnosis of heart disease in an in vitro setting. Typically, artificial hearts are constructed, in part, using rigid materials such as metals, and are externally powered using a compressed air pump system that directly pumps blood in order to generate flow. As these devices may be cumbersome, open to infection, subject to rejection by the patient's body and/or thrombosis, and expensive, the use of artificial hearts are typically meant to be temporary.

### SUMMARY

The present disclosure relates to artificial anatomical models and methods of making and using the same. More particularly, the subject matter of the present disclosure covers substantially anatomically correct models or subject-specific three-dimensional models of hearts with surrounding vessels, in which the artificial heart is capable of replicating intact cardiovascular functions and/or electrical activity for the purposes of treatment, diagnosis, and/or education. Additionally, the present disclosure covers the fabrication and use of the anatomical models, as well as apparatuses and systems for acquiring the anatomical data, synthesizing the physiology within such models and disseminating the synthesized results to the user.

An artificial organ and/or tissue (e.g., an artificial heart) constructed of elastomer materials may have a number of advantages over artificial organs and/or tissues that are constructed out of rigid plastics and metals. For example, in some implementations, an artificial heart constructed substantially entirely of an elastomer material may be actuated in such a manner that it replicates the same physical actions of a natural heart, including contraction and relaxation of atria and ventricles as well as the electrical activation and signaling to drive such actuation. When the actuation is induced in an elastic artificial heart having a substantially similar physiological function to a natural heart, a more natural blood flow through chambers and vessels may be achieved, providing sufficient supply of nutrients and oxygen to the body while reducing the occurrence of dangerous blood clots. Furthermore, in some implementations, bio-compatible elastomer materials are available that are resistant to rejection by a patient's immune system.

In general, according to one aspect, the subject matter of the present disclosure can be embodied in an artificial heart that includes an anatomically correct or patient-specific model of a natural heart, in which the model is substantially composed of an elastomer, and an actuation element, in which the actuation element is configured to, during operation of the artificial heart, cause the artificial heart to contract and relax.

Implementations the artificial heart can include one or more of the following features and/or features. For example, in some implementations, the actuation element includes multiple pneumatic channels embedded in walls of the artificial heart.

In some implementations, the actuation element includes multiple electrical conductors embedded in walls of the artificial heart. The actuation element further can include an electroactive polymer between a pair of electrical conductors. The electrical conductors can include electrical conductive polymer or ionic gels.

In some implementations, the elastomer includes silicone.

In some implementations, the artificial heart includes a sensor attached to the model, in which the sensor is configured to measure and/or modify different physiologic parameters associated with the artificial heart.

In general, according to another aspect, the subject matter of the present disclosure can be embodied in an artificial heart system that includes: an anatomically correct or patient-specific model of a natural heart, in which the model is substantially composed of an elastomer; an actuation element, in which the actuation element is configured to, during operation of the artificial heart, cause the artificial heart to contract and relax; and a power source coupled to the actuation element.

Implementations of the artificial heart system can include one or more of the following features and/or features of other aspects. For example, the actuation element can include a pneumatic tube, channels or passages embedded in walls of the artificial heart, and the power source can include a pump.

In some implementations, the actuation element can include multiple electrical conductors embedded in walls of the artificial heart, and the power source can include an electric voltage or current source. The actuation element further can include an electroactive polymer between a pair of electrical conductors. The artificial heart system can further include one or more magnets arranged to generate a magnetic field across the electrical conductors. The electrical conductors can include electrical conductive polymer or ionic gels.

In some implementations, the elastomer includes silicone.

In some implementations, the artificial heart system further includes a sensor attached to the model, in which the sensor is configured to measure and/or modify different physiologic parameters associated with the artificial heart.

In general, in another aspect, the subject matter of the present disclosure can be embodied in methods of fabricating an artificial heart, in which the methods including constructing data representing an anatomically correct or patient-specific representation of a natural heart; and fabricating an anatomically correct or patient-specific model of the natural heart based on the data in a three-dimensional printing device, in which the model is substantially composed of an elastomer.

The methods can include one or more of the following features and/or features of other aspects. For example, the methods can further include embedding an actuation element in one or more walls of the anatomically correct model. Embedding the actuation element can include embedding a pneumatic tube in the one or more walls of the anatomically correct model. Embedding the actuation element can include embedding multiple electrical conductors in the one or more walls of the anatomically correct model. Embedding the actuation element can include embedding an electro-active polymer in the one or more walls of the anatomically correct model.

In general, in another aspect, the subject matter of the present disclosure can be embodied in methods including obtaining, in a three-dimensional printing device, data representing an anatomically correct or patient-specific representation of a natural heart; fabricating an anatomically correct or patient-specific mold of the natural heart based on the data; filling the mold with an elastomer; curing the elastomer in the mold; and removing the mold from the cured elastomer, wherein the cured elastomer forms an anatomically correct model of the natural heart.

The methods can include one or more of the following features and/or features of other aspects. For example, in some implementations, the methods can further include embedding an actuation element in one or more walls of the anatomically correct model. Embedding the actuation element can include embedding a pneumatic tube in the one or more walls of the anatomically correct model. Embedding the actuation element can include embedding multiple electrical conductors in the one or more walls of the anatomically correct model. Embedding the actuation element can include embedding an electro-active polymer in the one or more walls of the anatomically correct model.

In general, in another aspect, the subject matter of the present disclosure can be embodied in methods of fabricating an artificial organ, in which the method includes: constructing data representing an anatomically correct or patient-specific representation of a natural organ; fabricating an anatomically correct or patient-specific model of the natural organ based on the data in a three-dimensional printing device, in which the model is substantially composed of an elastomer.

In general, in another aspect, the subject matter of the present disclosure can be embodied in methods of fabricating an artificial organ, in which the methods include: obtaining, in a three-dimensional printing device, data representing an anatomically correct or patient-specific representation of a natural organ; fabricating an anatomically correct or patient-specific mold of the natural organ based on the data; filling the mold with an elastomer; curing the elastomer in the mold; and removing the mold from the cured elastomer, in which the cured elastomer forms an anatomically correct model of the natural organ.

In general, in another aspect, the subject matter of the present disclosure can be embodied in an artificial heart that includes a patient-specific model of a natural heart ventricle, in which the model includes an elastomer wall, the elastomer wall defining an interior ventricle region, at least one fluid passage extending within the elastomer wall, and multiple inextensible fibers around an exterior of the elastomer wall. The artificial heart ventricle can further include a pneumatic coupling device, the pneumatic coupling device including an inlet and at least outlets, wherein the inlet is fluidly coupled to the at least one outlet, in which the at least one outlet is fluidly coupled to the at least one fluid passage extending within the elastomer wall. The elastomer wall can further include multiple bladders, in which adjacent bladders are separated from one another by an elongated gap region.

The details of one or more embodiments are set forth in the accompanying drawings and the description below. Other features and advantages will be apparent from the description and drawings, and from the claims.

### BRIEF DESCRIPTION OF THE DRAWINGS

FIG. 1A is a schematic that illustrates an example of an artificial heart.

FIG. 1B is a schematic that illustrates a perspective view of an artificial left ventricle.

FIG. 1C is a schematic illustrating a side view of the artificial ventricle depicted in FIG. 1B.

FIG. 1D is a schematic illustrating a top view of a cross-section taken at section A-A in FIG. 1C.

FIGS. 2-3 are schematics that illustrate examples of an artificial heart.

FIG. 4 is a schematic that illustrates an example of a system for fabricating artificial anatomical models.

FIG. 5 is a schematic illustrating a process for fabricating an artificial tissue or organ.

FIG. 6 is a schematic that illustrates an example of an artificial heart that includes a sensor.

### DETAILED DESCRIPTION

### Artificial Implants

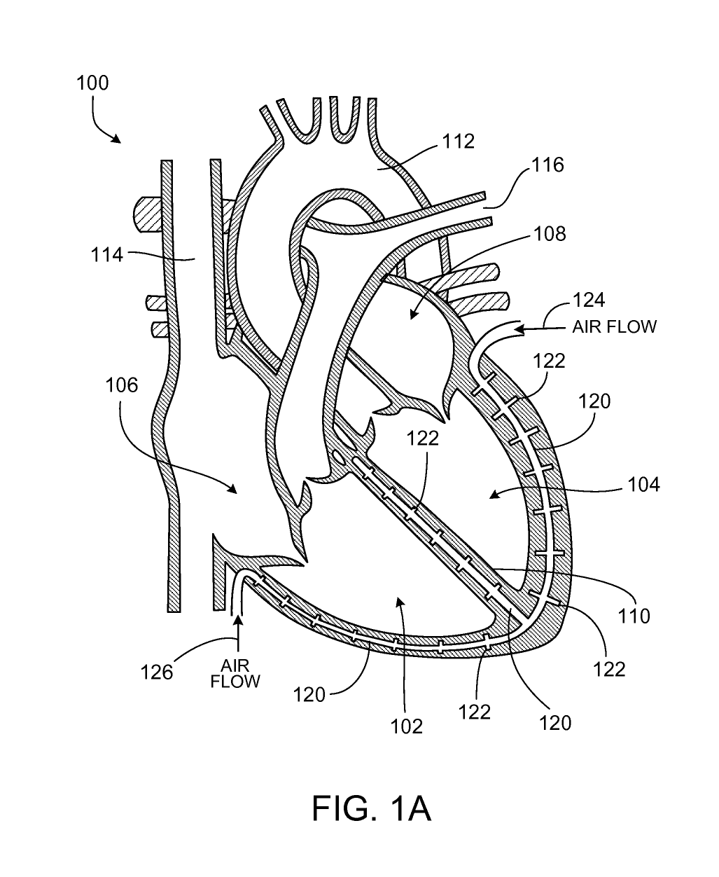

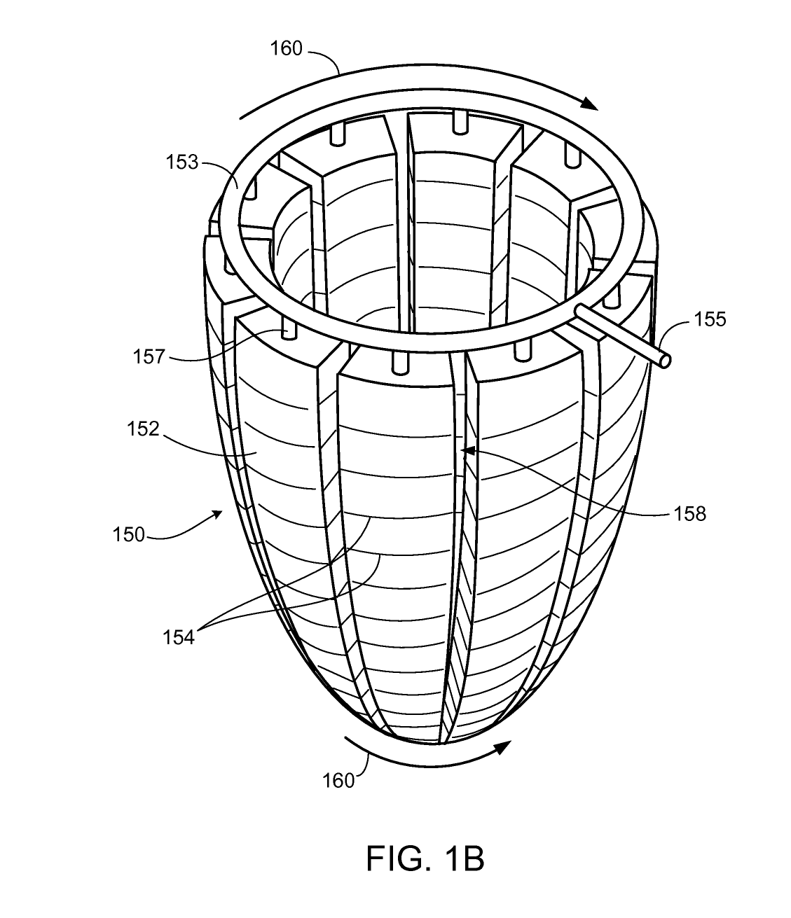

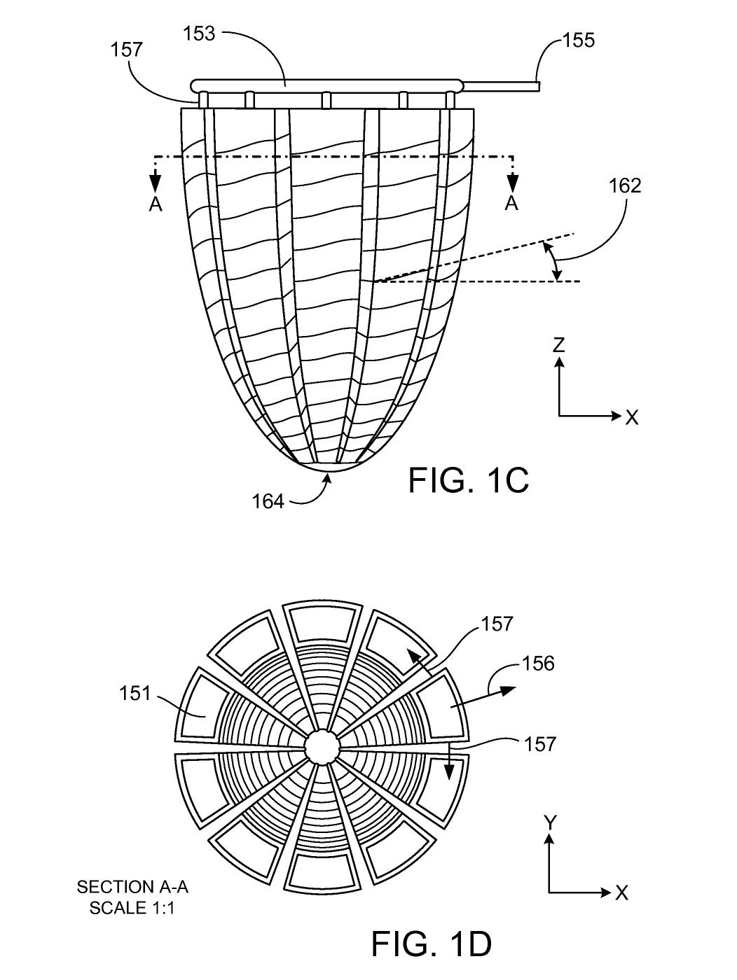

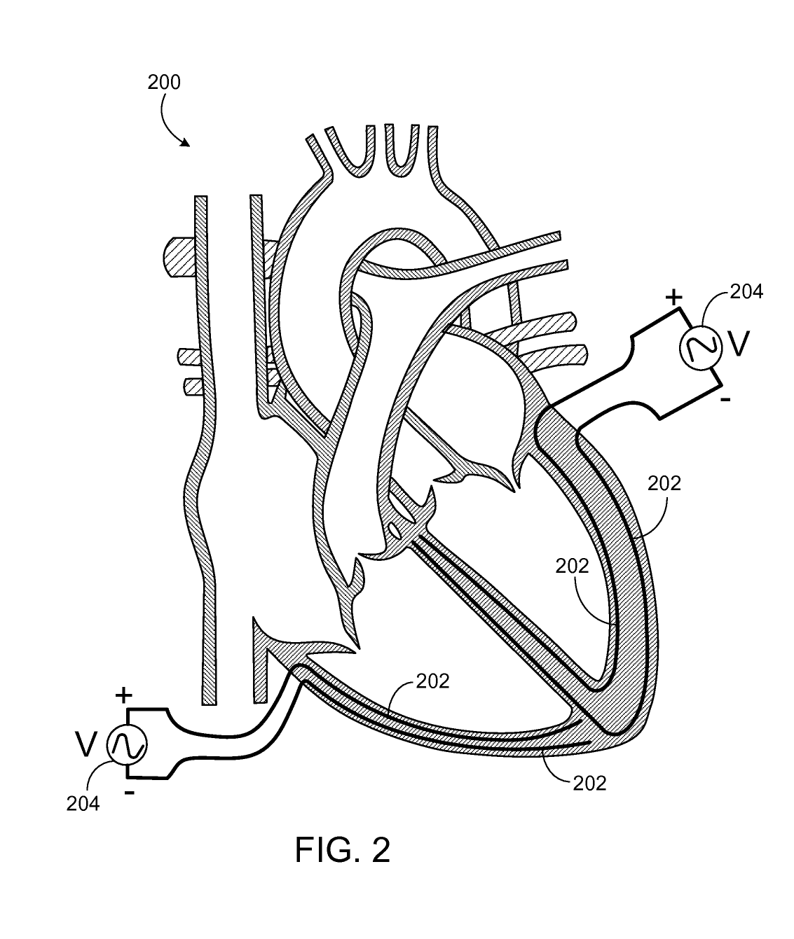

Various different mechanisms may be used to drive the myocardial deformation of an artificial heart constructed substantially entirely out of elastomer materials. For example, pneumatic, electrical or magnetic techniques may be employed to actuate the artificial heart and induce cardiovascular functions that replicate the natural behavior of a heart. The mechanical properties of the materials used to construct the artificial heart can be varied across different regions and sections of the artificial heart to achieve improved actuation performance and efficiency, and to better replicate the native heart anatomy, geometry, and physiology. FIG. 1A is a schematic that illustrates an example of an artificial heart 100 constructed out of an elastomer material, in which the artificial heart 100 is actuated using pneumatic regulation of air pressure in the channels inside the walls of the artificial heart. As shown in FIG. 1A, the artificial heart is a substantially anatomically correct subject-specific human heart having the same physiological function as a natural heart. A substantially anatomically correct subject-specific structure means that the features of the structure, including its actual size, shape, and contours, are determined and constructed to replicate the structure as it exists for a particular subject (e.g., a patient). The design and construction can be based on data obtained from the particular subject, such as, for example, medical imaging data of the structure. A substantially anatomically correct subject-specific structure contrasts with an anatomically correct natural structure, that has a shape, size and contours generally representative of how the structure would appear, but that are not intended to represent the actual features of the structure in a particular subject.

The heart 100 includes a right ventricle 102, a left ventricle 104, a right atrium 106, and a left atrium 108. The right and left ventricles are separated by a septum 110. The atria and ventricles of the heart 100 are configured to be substantially identical to the atria and ventricles of a natural heart, i.e., approximately the same volume/size and the same shape. The artificial heart 100 may optionally include additional components, such as portions of the aorta 112, the vena cava 114, pulmonary arteries 116, and coronary arteries and veins.

In order to replicate realistic ventricular contraction and relaxation in the artificial heart, a pneumatic system is incorporated into the elastomeric walls of the heart, in which the pneumatic system drives the deformation of the walls. The pneumatic system includes a network of fluid channels that may be placed in the artificial heart either during fabrication or at the post-processing stage. For example, as shown in FIG. 1A, the heart 100 includes fluid channels 120 in the outer walls and within the septum 110. The fluid channels may be designed and optimized by using a numerical solid or fluid-solid interaction solver according to the apparent motion observed in dynamic imaging data or during open-heart procedures in human and large animal studies. The layout and dimensions of the fluid channels can be varied to realize healthy or various abnormal motion patterns. For instance, in the example shown in FIG. 1A, the fluid channels 120 include a main channel and multiple narrow openings 122 spaced apart that are elongated in a first direction perpendicular to a direction of air-flow in the main channel. The fluid channels 120 include a first inlet 124 and other inlets 126 through which compressed air or other fluids (e.g., liquids such as saline) can be introduced. As the pressure of compressed air in the channels is oscillated, the elastic walls of the atria are forced to contract and relax, simulating the mechanical actions of a natural heart. The number and location of the inlets are chosen to maintain the regularity and fast adaptation of air pressure in the channels. The multiple inlets also enable the control of the filling and ejection of the two ventricles to balance the cardiac outputs for both sides of the heart. The compressed air may be provided using a pump external to the artificial heart 100. For example, in some implementations, the inlets of the artificial heart 100 may be coupled to tubing that, in turn, is coupled to a pneumatic pump/air compressor external to the patient. The compressor may include an electronic controller that directs the compressor to supply continuous or time-varying driving pressure to regulate the motion of heart contraction and relaxation.

In some implementations, in order to achieve pumping action that simulates the actual pumping of blood with a natural heart, the walls of the artificial organ or tissue (e.g., heart) may be modified in certain regions to enable natural heart-like contraction. In particular, the walls may be reinforced locally by using materials having a stiffness greater than the elastomer forming the wall. Alternatively, or in addition, inextensible fibers can be attached to the elastomer walls to locally constrain the elastomer displacement. In either case, the regions of the elastomer that are not covered by a stiff material or fiber will experience greater expansion than without the stiff materials in place.

As an example, FIG. 1B is a schematic that illustrates a perspective view of a glove-like flexible structure 150 that can be used as part or all of an artificial left-ventricle. FIG. 1C is a schematic illustrating a side view of the structure 150 and FIG. 1D is a top view of the structure 150 through section A-A. For clarity, the structure 150 is shown to have a general shape corresponding to approximately half of an ellipsoid. However, a subject (e.g., patient) specific artificial heart would have a shape, dimensions and contours that follow the anatomical variations of an actual heart. Although not shown, the cavity of the ventricle structure 150 would couple to a patient's aorta and left atrium at the top region near manifold 153.

The glove-like structure 150 includes multiple individual elastomer bladders 152 wrapped with inextensible fiber reinforcements 154. Each bladder 152 is hollow inside (e.g., see hollow regions 151 in the top view of FIG. 1D) or alternatively, at least fluid permeable to receive a fluid, such as air, that causes the elastomer walls to expand during use of the structure 150. That is, each bladder 152 acts like a balloon such that the bladder 152 inflates from the inside and the elastomeric walls of the bladder 152 expand outwardly. The bladders 152 are fixed to one another at a common point/area of the structure 150, e.g., at a bottom 164 of the structure. The internal openings of each bladder 152 can terminate at the common point or can be fluidly coupled to one another.

Each bladder 152 also is wrapped with inextensible fibers 154. By wrapping each bladder with inextensible fibers 154, the walls of the bladders 152 are constrained from deforming/expanding in the regions where the fibers are located. Depending on the arrangement of the fibers 154, the motion and direction of the expansion can be controlled. For instance, as shown in FIG. 1B, the fibers 154 are wrapped around each bladder 152 in a helical manner. As a result of the helical wrapping, the elastomer material of the walls expands both outwardly (in a direction 156 away from a longitudinal axis extending through center of the structure 150) and laterally (in a direction 157 approximately tangential to a ring surrounding the center longitudinal axis), causing a torsional motion 160 of the bladder walls 152. The orientation of the fibers 154 is not limited to a helical pattern and can include other patterns as well. For example, an additional set of fibers can be wrapped around each bladder 152 with an opposing helical direction, such that the fibers 154 crisscross at multiple locations. The orientation and density of the fibers may be modified to enhance certain motion modes, e.g. to increase torsion or increase expansion of the bladder walls. For example, the angle 162 of the inextensible fibers 154 with respect to a planar surface that extends tangential to the bottom 164 of the structure can be increased to increase the torsional motion upon expansion or the angle 162 can be decreased so that the structure 150 simply expands with less torsional motion or no torsional motion.

The inextensible fibers 154 can include, e.g., carbon/graphite fibers, reinforced carbon-carbon threads, carbon fiber reinforced polymer threads, or Kevlar® threads such as Kevlar® 49 quasi-unidirectional fabric, or other inextensible fibers. The inextensible fibers 154 can be situated in grooves formed on the outer walls of the bladders 152 to help align the fibers 154 along a helical pattern.

As shown in FIGS. 1B-1D, the each bladder 152 can be separated from an adjacent bladder 152 by a gap region 158. This gap region 158 allows the bladders space for expansion when inflated. The width of each gap regions 158 (i.e., the width of the space between adjacent bladders 152) can vary. For example, in some implementations, the maximum width can be between approximately 0.1 mm to approximately 4 mm (e.g., approximately 0.25 mm, 0.5 mm, 0.75 mm, 1 mm, 1.25 mm, 1.5 mm, 1.75 mm, 2 mm, 2.25 mm, 2.5 mm, 2.75 mm, 3 mm, 3.25 mm, 3.5 mm, or 3.75 mm). Other widths are also possible. The gap regions 158 can be filled with an elastomer material having a stiffness that is the same as the elastomer that forms the bladders 152. Alternatively, the gap regions 158 can filled with an elastomer having a stiffness that is less than the stiffness of the elastomer forming the bladders 152 to allow greater expansion of the bladders 152 into the gap regions 158. Furthermore, the material filling the gap regions 158 can form a water tight seal with the bladders 152.

Each of the openings within a bladder 152 of the artificial ventricle 150 can be coupled to a ring manifold 153. The ring manifold 153 is formed from plastic or other suitable biocompatible material. Fluid pressure (e.g., air pressure) from a pump system is coupled to an inlet 155 of the ring manifold 153 and then delivered to the openings within each bladder 152 through a coupler 157. During operation of the structure 150, the openings within each bladder 152 are filled with a fluid (e.g., air) that causes the elastomer of each bladder 152 to expand outwardly and in a direction partially determined by the constrictions (e.g., inextensible fibers) attached to the bladders' outer surfaces. The fluid then may be withdrawn to allow the structure 150 to relax again. This process of expansion and relaxation can be repeated so that the structure performs a pumping action.

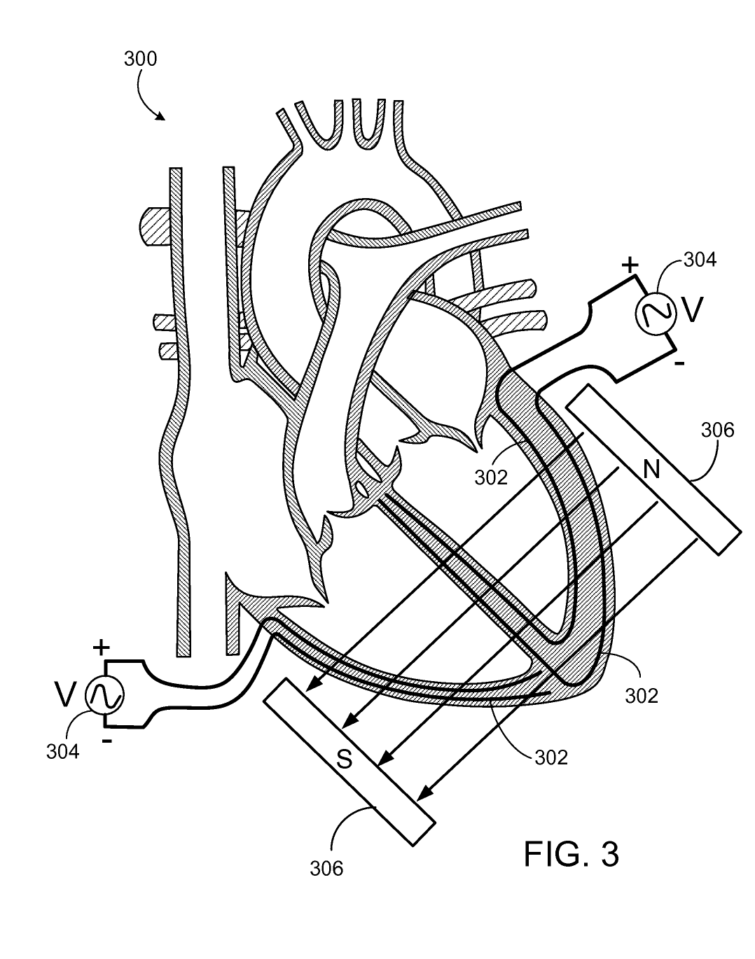

In some implementations, an electrical-based actuation mechanism is used to replicate realistic ventricular contraction and relaxation in the artificial heart. FIG. 2 is a schematic that illustrates an example of an artificial heart 200 constructed out of an elastomer material, in which the artificial heart 200 is actuated using electrical stimulation of electrically conductive materials. As in the example of FIG. 1, the artificial heart 200 has substantially the same physiological function as a natural heart. Identification of different heart features is omitted in FIG. 2 for clarity. In the present example, the wall of the heart 200 is formed of electroactive polymer. It includes dielectric deformable elastomer and electrical conductors 202, which are incorporated into the walls and septum of the heart 200 and are electrically coupled to one or more power sources 204. The electrical conductors 202 may be fabricated from any suitable conductive and flexible material (e.g., electrically conductive polymers or ionic gels) and may be placed in the artificial heart either during fabrication or at the post-processing stage. The electrical conductors are embedded in the elastomer and separated from the blood pool and other nearby tissues. The electrical conductors 202 displace due to electrostatic forces when a potential is applied across the positive and negative electrodes shown in FIG. 2. The expansion and contraction of the electro-active polymer in turn causes contraction and relaxation of the walls of the artificial heart's cavities to allow a pumping action. The power sources 204 may include one or more batteries, a DC-DC converter to elevate the voltage, and an electronic controller configured to adjust the amount of power delivered to regulate the motion of heart contraction and relaxation. In addition, the power sources 204 may be arranged within the patient or external to the patient. In some implementations, the artificial heart is provided with two power sources: an external power source that is used for long-term driving of the artificial heart 200 and a power source implanted within the patient as a backup in case the external power source ceases to provide power.

In some implementations, a magnetic-based actuation mechanism is used to replicate realistic ventricular contraction and relaxation in the artificial heart. FIG. 3 is a schematic that illustrates an example of an artificial heart 300 constructed out of an elastomer material, in which the artificial heart 300 is actuated using the application of magnetic fields to induce deformation of the materials forming the heart 300. As in the examples of FIGS. 1 and 2, the artificial heart 300 has substantially the same physiological function as a natural heart. Identification of different heart features is omitted in FIG. 3 for clarity. In the present example, electrical conductors 302 are incorporated into the walls and septum of the heart 300 and are electrically coupled to one or more power sources 304. The electrical conductors 202 may be fabricated from any suitable conductive material (e.g., electrically conductive polymers or ionic gels) and may be placed in the artificial heart either during fabrication or at the post-processing stage. The power sources 304 and electrical conductors 302 may be configured such that an electric current travels through the electrical conductors 302. During operation of the artificial heart 300, a magnetic field may be generated such that the field extends across the current-carrying electrical conductors 302. As a result of the current traveling in the presence of the magnetic field, the electrical conductors 302 experience a Lorentz force that causes the electrical conductors 302 to move. If the force is large enough and regulated, the movement of the electrical conductors 302 will deform the ventricular walls inducing contraction or relaxation of the heart. To generate the magnetic field, a series of bio-compatible magnets 306 may be implanted with the heart 300 into the patient, e.g., adjacent to the sides of the ventricles. Alternatively, the magnets may be arranged externally to the patient. The magnets may include electromagnets or permanent magnets. In certain implementations, the power source and/or magnets are coupled to an electronic controller that controls the current through the electrical conductors 302 and/or the magnitude of the magnetic field in order to regulate the motion of heart contraction and relaxation.

Though the foregoing examples pertain to substantially anatomically correct replicas of a subject-specific heart, the same materials and methods can be applied to other substantially anatomically correct subject-specific structures. For example, substantially anatomically correct subject-specific replicas of myocardial tissue and/or surrounding soft tissue structures (e.g., pericardium, fat) can be fabricated out of elastomer materials, including electroactive polymers. In some implementations, the replica represents only portions of a heart including, for example, only one, two or three of the main heart chambers. In some implementations, the replica corresponds to a vascular and/or endovascular implants including, but not limited to, the aorta, coronary arteries, left atrial appendage, pulmonary arteries, pulmonary veins and other systemic veins, bypass grafts, and/or stents. The implants may be modified from subject-specific include altered cardiac anatomy and/or physiology.

To accurately replicate the physiological movement and actions of a subject-specific tissue or organ (e.g., a heart including, but not limited to, ventricular contraction with physiologically appropriate cardiac output and flow), the artificial structure is fabricated to have substantially the shape, size and physical features of a subject-specific tissue or organ. That is, the shape, size, contour, and surface features of the artificial structure are fabricated to match a natural structure (such as a subject's own heart) as close as possible, where the matching accuracy of the replica is limited by the anatomical data representative of the natural heart (e.g., the minimum resolution of digital images of the natural heart) and the fabrication process for constructing the artificial heart (e.g., the resolution and uniformity of the fabrication process). Because the anatomical data used to fabricate the artificial heart can be located either locally with respect to the fabrication system (e.g., the data can be stored in memory on the same computer system used to control the fabrication process) or remotely with respect to the fabrication system (e.g., the data can be stored on a different computer system from the system used to control the fabrication process) and because the fabricated artificial heart can be used locally or transferred to a remote site, systems enabling different workflows of data and fabricated artificial heart transmission can be required.

Different techniques may be used to fabricate an artificial tissue or organ (e.g., a heart) such that is anatomically correct in three-dimensions. In some implementations, a computer system is provided, in which the system is configured to receive and/or store in memory anatomical data representative of the tissue or organ to be constructed (e.g., digital image data), construct models of the artificial structure in digital format based on the anatomical data, optionally store the constructed models in the memory, and then fabricate physical models based on the digital representation using, e.g., 3D printing techniques. Once fabricated, the physical model can be provided to a user.

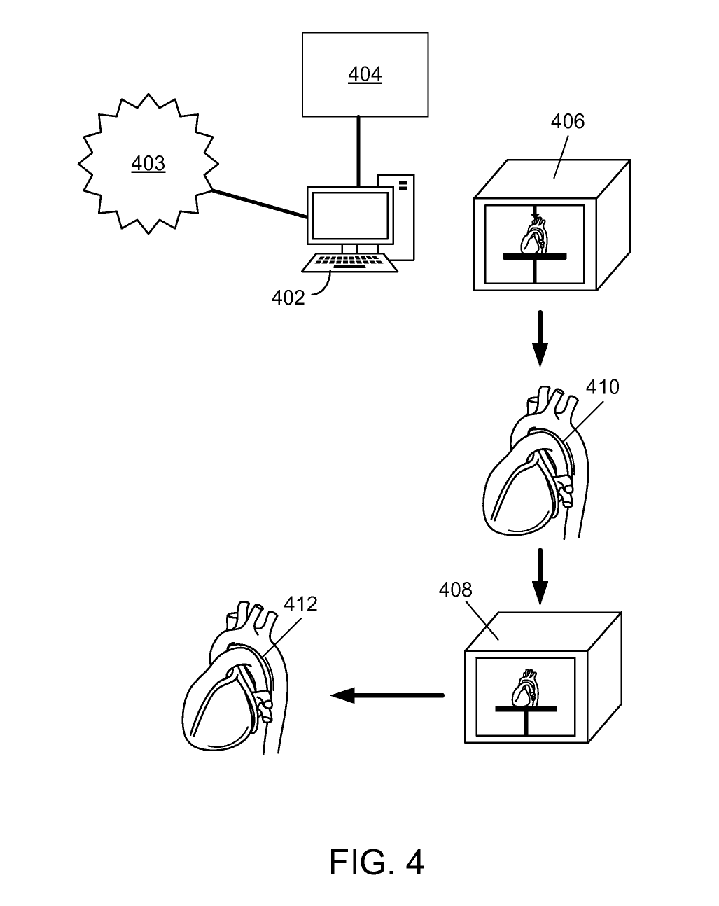

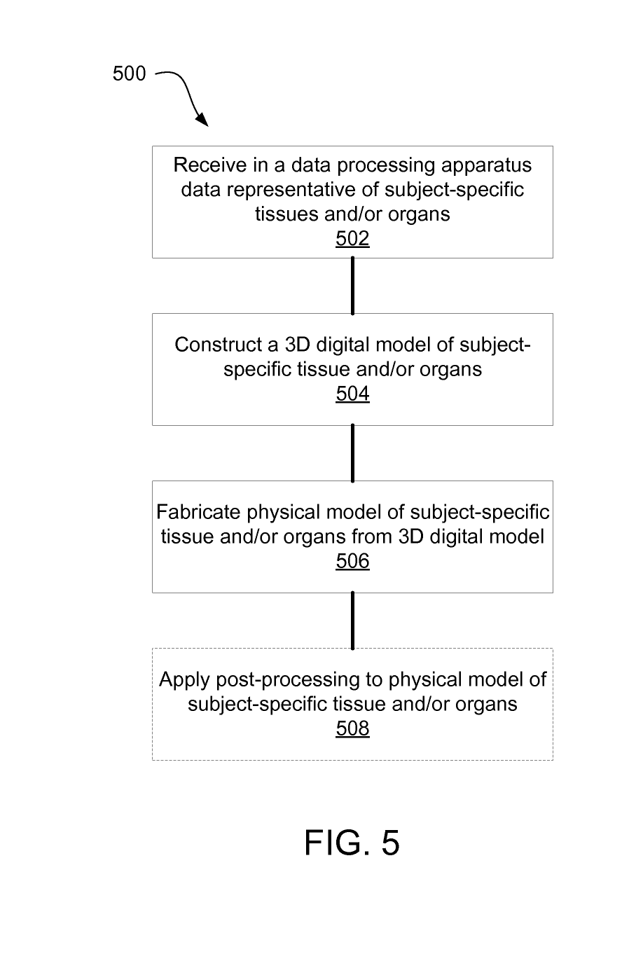

FIG. 4 is a schematic that illustrates an example system 400 for fabricating an artificial heart according to the present disclosure. The system 400 and process associated with system 400 can be used to fabricate other artificial tissues and/or organs as well. The system 400 includes data processing apparatus 402 having at least one processor. The data processing apparatus can include one or more computers 402 each having their own memory or shared memory. The data processing apparatus 402 can be configured to operate as a web server that hosts a user web portal through which users can upload anatomical data (e.g., digital image data such as CAD files or medical digital image data obtained from medical imaging devices). Accordingly, the data processing apparatus 402 can be coupled to a computer network 403, such as a local area network, a wide area network, and/or larger networks, including the Internet. Alternatively, or in addition, the data processing apparatus 402 can be electronically coupled to one or more medical imaging devices 404 (e.g., computed tomography (CT) scanning device, positron emission tomography (PET) scanning device, magnetic resonance imaging (MRI) device, ultrasound imaging device, intravascular ultrasound device, and/or optical coherence tomography device, among others) to receive the imaging data from the medical imaging device. The data processing apparatus 402 can be configured to construct an anatomically correct digital model of the artificial heart based on the digital data received at the data processing apparatus 402. For example, the data processing apparatus 402 can include a computer program that converts medical imaging data into a 3D digital model. The data may be in various file formats such as, for example, medical imaging datasets or computer aided drawing (CAD) files, or other suitable file format, and can include, for example, 2D or 3D images. Converting the medical imaging datasets into a 3D digital model can include, for example, using the data processing apparatus 402 to perform segmentation of the medical images, geometric modeling (e.g., intersection removal between images, mesh smoothing, shelling, among other geometric modeling actions), generation of support structures, tool path generation, and development of printing control algorithms following the tool path. The foregoing functions can be implemented in software running on the data processing apparatus 402 or in combination with special purpose logic circuitry, e.g., an FPGA (field programmable gate array) or an ASIC (application specific integrated circuit). Using the digital model, the computer(s) 402 instructs a 3D printer 406 to fabricate in an elastomeric material a full-scale 3D model 410 of the heart, including cavities to allow blood flow through the different chambers and vessels. Once printed, the physical model is a 3D replication of the anatomical structure. A post-processing machine 408 also can be included as part of the system 400, in which the post-processing machine 408 is used to modify the artificial heart so that it can be actuated according to the desired actuation mechanism (e.g., using air pressure, electro-mechanical actuation, or magnetic actuation). For example, the post-processing machine 408 can be used to add components to the physical model (e.g., adding a pneumatic ring manifold to an artificial heart such as manifold 153 from FIG. 1B, adding inextensible fibers similar to fibers 154 from FIG. 1B, adding electrical conductors and electrodes, and/or adding magnetic materials such as permanent magnets). The post-processing can be done manually or using a specially configured system to add the components to the physical model. In some implementations, the 3D digital model design can be modified by the data processing apparatus 402 prior to fabrication. For instance, the digital model can be modified from a subject-specific model to include openings for fluid channels, electrically conductive materials, and/or magnetic materials, such that when the 3D model is physically fabricated using a 3D printer, it is formed to include the openings without the need for post-processing. For an artificial heart constructed to implement realistic pumping action, the outer walls of the model can be modified to include grooves for placement and alignment of the inextensible fibers. In some implementations, the subject-specific heart is fabricated without any modification to incorporate an actuation mechanism. The final printed heart 412 can then be transferred to the end-user with or without being modified to include the actuation mechanism. FIG. 5 is a schematic illustrating a process 500 for fabricating an artificial tissue or organ using the system 400 of FIG. 4. In a first step (502), data representative of an artificial structure, such as a patient's tissue or organ, is received in a data processing apparatus. The data can include digital image data that is uploaded to the data processing apparatus through, for example, a web portal hosted by the data processing apparatus. Alternatively, the data can be downloaded, wirelessly or through a network connection, to the data processing apparatus 402 from a medical imaging device 404 or from a memory device (e.g., flash memory stick, DVD, CD or other appropriate memory device). The data can include digital images representative of an organ or tissue that is specific to a particular subject (e.g., a patient). Using the digital image data, the data processing apparatus 402 constructs (504) a 3D digital model of the artificial structure (e.g., heart, artery, vein or other structure) in a file format for a 3D printer (e.g., STereoLithography file format (STL), OBJ format, or Additive Manufacturing File Format (AMF), or X3D, among other file formats). This step can optionally include modifying the 3D digital model to include features for allowing actuation of the artificial structure (e.g., the inclusion of fluid channels and other openings within the walls of a heart, or other openings for placing electrically conductive or magnetic materials). The data describing the 3D digital model of the artificial structure then is passed from the data processing apparatus to the 3D printer 406, which uses the data describing the 3D digital model to fabricate (506) a physical model of the artificial structure. Construction of the physical model may include fabricating different regions or areas of the model to have elastomers with different mechanical properties (e.g., by varying the density of the elastomer that is printed). Alternatively, or in addition, this can include varying the type of elastomer for different regions or areas of the model (e.g., using electro-active polymers for portions of the structure that will be actuated by applying an electric potential or magnetic field, while using non-electro-active polymers for other areas of the structure). An optional post-processing step (508) includes modifying the artificial structure to include, for example, features necessary to actuate the structure. This can include creating fluid channels and other openings within walls of the structure, creating openings within the walls of the structure for receiving electrically conductive and/or magnetic materials, and placing such electrically conductive and/or magnetic materials within the openings. The artificial structure, once fabricated, then can be provided to the end-user for the desired use (e.g., therapeutic, diagnostic, or educational).

In general, 3D printing technologies are based on an additive manufacturing process but differ in ways of depositing layers of materials, which may include, but are not limited to, stereolithography fused deposition modeling, selective laser sintering and laminated object manufacturing. The dimensions and arrangement of the different parts of the heart can be designed manually or obtained with or without the aid of cardiac imaging data. For instance, images of the heart may be obtained using computed tomography (CT) scanning, positron emission tomography (PET) scanning, magnetic resonance imaging (MRI), ultrasound imaging, intravascular ultrasound, and/or optical coherence tomography, among others. Other imaging techniques also may be used to obtain images of the various features, both internal and external, of a heart. Such images may be used to obtain a design of a normal functioning heart or a design of a diseased or defective heart. A subject-specific heart model fabricated based on a diseased or defective heart may be used for educational and/or diagnostic and/or therapeutic purposes.

In some implementations, the artificial implants are constructed without all of the components of a corresponding natural organ or tissue. For example, an artificial heart may be constructed to include all of the features of a natural human heart (e.g., atria, ventricles, surrounding soft tissue structure) but not include the valves that separate the chambers of the heart. Such components may be fabricated separately or left out of the artificial heart entirely. Another example is the artificial heart may only correspond to the left side of the heart or the two ventricles instead of all four chambers. These partial implementations may be advantageous if some functional chambers of the patient are left intact; as an example, when implanting artificial hearts.

In some implementations, the artificial implant (e.g., heart) model design is modified from the subject-specific (e.g., patient-specific) design to incorporate the actuation mechanism. For example, similar to FIG. 1B, the design of an artificial left-ventricle can be modified from a subject-specific left ventricle to include gap regions to allow expansion of elastomer material in a desired direction. In addition, openings within the elastomer material can be designed to receive fluid pressure for expansion of the elastomer. In addition or as an alternative, grooves can be designed into the outer walls of the elastomer/ventricle to allow placement of inextensible fibers after fabrication, in which the inextensible fibers serve to constrict expansion of the elastomer material in predetermined directions so that the artificial heart behaves similar to a natural heart during expansion and contraction. These features (e.g., gap regions, openings within elastomer, grooves, among other features) can be introduced after the 3D digital model is constructed based on medical imaging data, but before the physical 3D model is fabricated using a 3D printer. After the physical 3D model is fabricated, the individual bladders 152 can be wrapped with inextensible fibers 154 (e.g., by following a groove formed in the bladders walls of the physical model). The gap regions 158 then can be filled with an elastomer (e.g., silicone or polyurethane into a mold that fixes the elastomer in a region between the bladders 152) to seal the bladder regions 152 together, after which the elastomer is cured to solidify. When placed within a patient, the top of the structure 150 then can be coupled to the aorta and left atrium. The top of the bladders 152 also can be coupled to a pneumatic manifold 153 through which fluid pressure (e.g., air pressure) may be introduced into each of the bladders 152 to cause expansion.

Though the examples of artificial anatomical structures disclosed in various implementations pertain to an artificial heart, other anatomical structures can also be fabricated using the techniques disclosed herein for diagnosis, therapeutic and/or educational purposes. For instance, in addition to features such as the myocardial tissue and surrounding soft tissue structures (e.g., pericardium, fat), and the four main heart chambers, the system 400 may be used to fabricate vascular and/or endovascular implants as well including, but not limited to, the aorta, coronary arteries (such as the epicardial coronary arteries), left atrial appendage, pulmonary arteries, pulmonary veins and other systemic veins, bypass grafts, and/or stents. Endovascular implants, such as stents or bypass grafts, may be fabricated and deployed to synthesize altered cardiac anatomy and/or physiology. Depending on the anatomy of interest and physiology to be synthesized, some of these components may be optional to save cost and time to fabricate. The shape, size and physical features of the structures can be fabricated to either replace or serve as a three-dimensional model for in vitro diagnostic testing. In the case of fabricated vessels (e.g., arteries, veins), the vessels can be connected to a pulsatile flow system that replicates fluid flow through the vessels (e.g., velocity and pressure), as would be achieved through normal cardiac contraction. These methods can be performed through pneumatic, electrical and magnetic means, similar to that of heart contraction, as detailed herein. Other anatomical models that can be fabricated by the system 400 for diagnosis or educational purposes include, but are not limited to, organs such as the brain, liver, kidney, pancreas, intestines, endocrine glands, and skeletal muscle. As with the heart, any of the foregoing models may be subject-specific, in which the design of the model is obtained by imaging the corresponding anatomical part of a subject (e.g., patient) using imaging data, such as CT, PET, MRI, ultrasound or intravascular ultrasound, and/or optical coherence tomography for model inputs.

As an alternative to fabricating the physical models of the anatomical structures using 3D printing technology directly, the physical models can, in some implementations, be fabricated using mold-casting techniques, in which the mold is made by the aforementioned 3D printing techniques. Of course, techniques other than 3D printing can be used to fabricate the mold itself. Once the physical mold is fabricated, the elastomer is added to the mold and cured to form the model. The cured model is removed from the mold or the mold is dissolved in a suitable chemical.

As explained above, the materials used for the physical models of anatomical structures may include elastomers. For artificial implants such as artificial hearts, the elastomers are sufficiently flexible to deform and return to their original shape for the purpose of, e.g., performing contraction and relaxation similar to a natural heart. Materials for the elastomer include, but are not limited to, unsaturated rubbers and saturated rubbers, polyethers, polyester urethanes, and polyether polyester copolymers. An example of an elastomer that is biocompatible and suitable for use as an implantable artificial anatomical models constructed according to the present disclosure includes silicone, particularly silicone rubber materials that have been modified to increase tear strength and fatigue resistance. The soft electric conductors used in the electrical-based and magnetic-based actuation mechanisms may include electric conductive silicone, conductive carbon or metal grease, or conductive hydrogel.

In some implementations, it is not necessary for the material of the anatomical model to include an elastomer. For instance, if the anatomical model is used for educational or diagnostic purposes, the material forming the model may be stiff rather than flexible or compliant. Examples of materials that may be used for forming stiff models include, but are not limited to, polystyrenes, polylactide and polyvinyl chlorides. Whether the material used is elastic or stiff, the models can be fabricated to be either transparent or opaque depending on the materials used and the end-use applications intended for the physical models. In some implementations, the material used is colored so as to aid distinguishing different parts of the anatomical model. For example, in the case of a heart model, the material forming the chambers may be red whereas the material forming the pulmonary arteries may be blue. In some implementations, the material used is transparent so that the flow circulating in the chambers and through valves can be visualized and magnified with optical systems, such as particle image velocimetry.

In some implementations, the anatomical model formed using the 3D printing or mold techniques serves as a scaffold on which partial or full tissue engineering is performed. In partial or full tissue engineering, live cells and/or organs from the same patient may be affixed or integrated with the fabricated scaffold. Accordingly, when used as an implant, instances of transplant rejection by the patient may be reduced since the patient's body comes into contact with the engineered tissue and not the scaffold. Similarly, the use of engineered tissue may reduce the occurrence of clots in cardiac and vascular implants. In some implementations, the scaffold is formed from a non-degradable material. In other implementations, the scaffold itself may be constructed of materials that are biologically degradable. Once the live cells and/or organs are affixed to the degradable scaffold, the scaffold is naturally removed (e.g., through dissolving the scaffold), leaving the engineered tissue in place. Integrated cells and/or organs may include pluripotent stem cells as well as fully differentiated cell types.

In some implementations, the anatomical models may be used as heterotopic transplants, i.e., the models serve to augment the functionality of an organ or tissue existing in the patient. In other implementations, the anatomical models may be used as orthotopic transplants, i.e., the models serve to replace the existing organ or tissue in the patient.

### Diagnostic Applications

As explained above, the physiological models fabricated according to the present disclosure may be used for diagnostic purposes, such as studying a diseased or defective anatomical structure for evaluation of the structure's functionality and/or evaluation of the effectiveness of potential treatment options. In some implementations, the techniques disclosed herein may be used to generate realistic cardiac electrophysiology models. For instance, an artificial heart constructed substantially entirely of an electrically conductive and elastic material may be used to replicate the electrical stimulation and blood flow. To replicate blood flow, a circulation system is constructed to visualize or measure flow characteristics within the three-dimensional physical artificial heart models. In addition, other components may be included to sense/detect abnormalities under different physiological conditions, and compare potential outcomes between alternative treatment options, or evaluate safety and efficacy of existing or new devices.

In some implementations, in order to replicate the blood flow in the physical model, the circulation system includes at least the fabricated heart model, a pulsatile or steady flow pump, a reservoir, connection pipes, and other control and monitoring apparatus, the combination of which are able to replicate realistic flow conditions in the chambers, through the valves and in the vessels. A control apparatus having an electronic processor may be coupled to the flow pump to modulate the waveform of the flow pumping from and/or through the heart according to defined heart rates, stroke volumes, and ejection fraction. The control apparatus also may include control valves (serving as equivalent resistances in circuits) and closed air tanks (serving as equivalent capacitances in circuits) that modulate the outflow boundary conditions of the distal coronary and systemic circulation. All of the flow leaving the outlets can be collected in the reservoir and returned to the flow pump. The system also may include internal or external transducers and meters that allow monitoring of flow, pressure, velocity, stress and resistance at any location of the circulation (e.g., within the artificial heart and/or elsewhere in the circulation). With the heart made with materials of spatially-varying porosity, the perfusion in the heart tissue can be evaluated under different physiologic conditions when imaging with contrast enhanced flow.

In order to replicate realistic cardiac electrophysiology, an electrical system is design to generate electrical stimulation and conduction in the physical model with electrical conductive materials, which can be either stiff or flexible. The resistance of the conductive materials and the induced voltage is selected to replicate the electric currents and voltages observed in humans and large animals. The electrical activities are then measured by electrocardiography, voltage mapping or other techniques to elucidate the etiologies, manifestations and mechanisms of altered electrical conduction. The system to replicate electrophysiology can also be coupled with a system to replicate ventricular contraction to study the interaction between the electrical and mechanical function of the heart. A comprehensive system configuration may have at least these three components integrated and can thus be able to interconnect ventricular contraction, blood flow, and electrophysiology in a manner that simulates how a whole heart functions.

With the above construction, variations in the models and systems can be made in the applications to diagnose cardiovascular abnormalities under different physiological conditions, and compare potential outcomes among alternative treatment strategies; as well as evaluate safety and efficacy of existing and new devices or artificial organs. One example is cardiac output that can be measured in subject-specific physical models to determine the level of systemic flow. Another example is the mechanical properties (e.g. stiffness, thickness and porosity) of the myocardium or coronary artery wall can be varied to study ventricular motion and perfusion, as well as the motion-induced coronary plaque deformation and/or rupture.

With the above methods, sensors can be attached to measure and/or applied to modify different physiologic parameters. An example of this can be the application of accelerometers to the constructed artificial heart models, which can sense movement, motion, and change in direction. A feedback control loop can be constructed within this model so that the sensed changes can evoke a corrective response in driving mechanisms (such as flow or ventricular contraction) to alter the artificial heart function and meet physiologically-realistic needs in a timely fashion. Another example is the application of pressure sensors in both ventricles to monitor and balance the contractility and cardiac output of the left and right sides of the circulation.

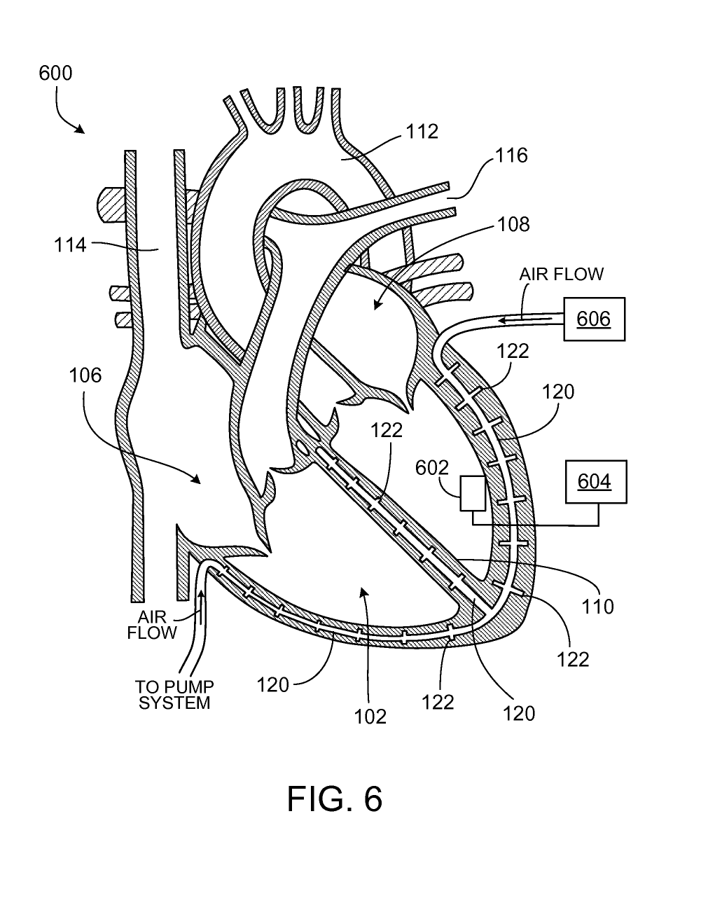

FIG. 6 is a schematic illustrating an example of an artificial heart 600 fabricated according to the present disclosure. The heart 600 is similar to the structure of FIG. 1A or 1B in which the heart is configured to induce pumping of blood through contraction and relaxation based on a pneumatic operation where a fluid (e.g., air) is introduced and withdrawn from openings within the walls of the artificial heart. The same reference numerals in FIG. 1A that are used in FIG. 6 refer to the same features. The heart 600 includes a sensor 602 (e.g., an accelerometer or pressure sensor) fixed to an inner surface of a wall of one of the ventricles in the heart. The sensor 602 can be electronically coupled (e.g., through a wire or wirelessly) to an electronic processor 604 located elsewhere in the patient's body or external to the patient. The electronic processor 604 can be electronically coupled to a pump system 606 that provides the fluid flow that causes the contraction and relaxation of the artificial heart walls. During operation of the heart 600, the sensor 602 can measure, e.g., flow or pressure, and provide an electrical signal to the electronic processor 604, which, in turn, adjusts the rate of fluid pumping to the heart 600 based on the signal. As a result, the contraction and relaxation of the artificial heart, and thus the cardiac output of the heart 600, can be adjusted based, in part, on the measurements from the sensor 602. Though the implementation shown in FIG. 6 applies to an artificial heart in which a pneumatic actuation method is used to cause contraction and relaxation, sensors also may incorporated into other artificial hearts where the actuation method is different (e.g., based mechanical expansion of electroactive polymers or through the generation of a Lorentz force that causes the electrical conductors, and thus the elastomeric walls of the heart to move) and/or where a different structure (e.g., artery, vein, among others) has been artificially fabricated according to the present disclosure.

Certain implementations and functional operations of the present disclosure provided herein (e.g., the construction of a digital 3D model of a subject-specific structure based on imaging data) can be realized in digital electronic circuitry, or in computer software, firmware, or hardware, including the structures disclosed in this specification and their structural equivalents, or in combinations of one or more of them. Implementations of the invention can be realized as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this disclosure can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Implementations of the invention can be realized in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this disclosure contains many specifics, these should not be construed as limitations on the scope of the disclosure or of what may be claimed, but rather as descriptions of features specific to particular implementations of the disclosure. Certain features that are described in this disclosure in the context of separate implementations can also be provided in combination in a single implementation. Conversely, various features that are described in the context of a single implementation can also be provided in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications may be made without departing from the spirit and scope of the invention. Accordingly, other implementations are within the scope of the invention.

What is claimed includes:

1. An artificial heart comprising:
an anatomically correct or patient-specific model of a natural heart, wherein the model is substantially composed of an elastomer; and
an actuation element, wherein the actuation element is configured to, during operation of the artificial heart, cause the artificial heart to contract and relax.

2. The artificial heart of claim 1, wherein the actuation element comprises a plurality of pneumatic channels embedded in walls of the artificial heart.

3. The artificial heart of claim 1, wherein the actuation element comprises a plurality of electrical conductors embedded in walls of the artificial heart.

4. The artificial heart of claim 3, wherein the actuation element further comprises an electroactive polymer between a pair of electrical conductors.

5. The artificial heart of claim 3, wherein the electrical conductors comprise electrical conductive polymer or ionic gels.

6. The artificial heart of claim 1, wherein the elastomer comprises silicone.

7. An artificial heart system comprising:
an anatomically correct or patient-specific model of a natural heart, wherein the model is substantially composed of an elastomer;
an actuation element, wherein the actuation element is configured to, during operation of the artificial heart, cause the artificial heart to contract and relax; and
a power source coupled to the actuation element.

8. The artificial heart system of claim 7, wherein the actuation element comprises a pneumatic tube embedded in walls of the artificial heart, and the power source comprises a pump.

9. The artificial heart system of claim 7, wherein the actuation element comprises a plurality of electrical conductors embedded in walls of the artificial heart, and the power source comprises an electric voltage or current source.

10. The artificial heart system of claim 9, wherein the actuation element further comprises an electroactive polymer between a pair of electrical conductors.

11. The artificial heart system of claim 9, further comprising one or more magnets arranged to generate a magnetic field across the electrical conductors.

12. The artificial heart system of claim 9, wherein the electrical conductors comprise electrical conductive polymer or ionic gels.

13. The artificial heart system of claim 7, wherein the elastomer comprises silicone.

14. A method of fabricating an artificial heart, the method comprising:
constructing data representing an anatomically correct or patient-specific representation of a natural heart;
fabricating an anatomically correct or patient-specific model of the natural heart based on the data in a three-dimensional printing device, wherein the model is substantially composed of an elastomer.

15. The method of fabricating an artificial heart according to claim 14, further comprising embedding an actuation element in one or more walls of the anatomically correct model.

16. The method of fabricating an artificial heart according to claim 15, wherein embedding the actuation element comprises embedding a pneumatic tube in the one or more walls of the anatomically correct model.

17. The method of fabricating an artificial heart according to claim 15, wherein embedding the actuation element comprises embedding a plurality of electrical conductors in the one or more walls of the anatomically correct model.

18. The method of fabricating an artificial heart according to claim 15, wherein embedding the actuation element comprises embedding an electro-active polymer in the one or more walls of the anatomically correct model.

19. A method of fabricating an artificial heart, the method comprising:
obtaining, in a three-dimensional printing device, data representing an anatomically correct or patient-specific representation of a natural heart;
fabricating an anatomically correct or patient-specific mold of the natural heart based on the data;
filling the mold with an elastomer;
curing the elastomer in the mold; and
removing the mold from the cured elastomer, wherein the cured elastomer forms an anatomically correct model of the natural heart.

20. The method of fabricating an artificial heart according to claim 19, further comprising embedding an actuation element in one or more walls of the anatomically correct model.

21. The method of fabricating an artificial heart according to claim 19, wherein embedding the actuation element comprises embedding a pneumatic tube in the one or more walls of the anatomically correct model.

22. The method of fabricating an artificial heart according to claim 19, wherein embedding the actuation element comprises embedding a plurality of electrical conductors in the one or more walls of the anatomically correct model.

23. The method of fabricating an artificial heart according to claim 19, wherein embedding the actuation element comprises embedding an electro-active polymer in the one or more walls of the anatomically correct model.

24. A method of fabricating an artificial organ, the method comprising:
constructing data representing an anatomically correct or patient-specific representation of a natural organ;
fabricating an anatomically correct or patient-specific model of the natural organ based on the data in a three-dimensional printing device, wherein the model is substantially composed of an elastomer.

25. A method of fabricating an artificial organ, the method comprising:
obtaining, in a three-dimensional printing device, data representing an anatomically correct or patient-specific representation of a natural organ;
fabricating an anatomically correct or patient-specific mold of the natural organ based on the data;
filling the mold with an elastomer;
curing the elastomer in the mold; and
removing the mold from the cured elastomer, wherein the cured elastomer forms an anatomically correct model of the natural organ.

26. The artificial heart of claim 1, further comprising a sensor attached to the model, wherein the sensor is configured to measure and/or modify different physiologic parameters associated with the artificial heart.

27. The artificial heart system of claim 7, further comprising a sensor attached to the model, wherein the sensor is configured to measure and/or modify different physiologic parameters associated with the artificial heart.

28. An artificial heart comprising:
patient-specific model of a natural heart ventricle, wherein the model comprises an elastomer wall, the elastomer wall defining an interior ventricle region;
at least one fluid passage extending within the elastomer wall; and
a plurality of inextensible fibers around an exterior of the elastomer wall.

29. The artificial heart of claim 28, further comprising a pneumatic coupling device, the pneumatic coupling device comprising an inlet and at least outlets, wherein the inlet is fluidly coupled to the at least one outlet, and wherein the at least one outlet is fluidly coupled to the at least one fluid passage extending within the elastomer wall.

30. The artificial heart of claim 28, wherein the elastomer wall comprises a plurality of bladders, wherein adjacent bladders are separated from one another by an elongated gap region.
